(19) United States
(12) Patent Application Publication
(10) Pub. No.: US 2003/0138790 A1
(43) Pub. Date: Jul. 24, 2003

(54) Dynamic sequencing by hybridization

(76) Inventors: Andrea Schlauersbach, Darmstadt (DE); Cord F. Stahler, Weinheim (DE); Peer F. Stahler, Mannheim (DE); Michael Baum, Heidelberg (DE); Manfred Muller, Munchen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/130,288

(22) PCT Filed: Nov. 29, 2000
(86) PCT No.: PCT/EP00/11978

(30) Foreign Application Priority Data
Nov. 29, 1999 (DE) 199 57 320.4

(51) Int. Cl. C12Q 1/68

(52) U.S. Cl. 435/6

(57) ABSTRACT

The invention relates to a method for sequencing nucleic acids using carrier chips that contain polymer probes, which are constructed of nucleotides and/or nucleotide analogs and which permit a specific binding with nucleic acids present in the sample. The method is dynamically carried out in a number of cycles, whereby the sequence information obtained from a preceding cycle is used for modifying carrier-bound probes in the subsequent cycle.

### DESCRIPTION

The invention relates to a method for sequencing nucleic acids using support chips which contain polymer probes constructed from nucleotides or/and nucleotide analogs and which allow specific binding to nucleic acids present in a sample. The method is carried out dynamically in a plurality of cycles, with the sequence information obtained from a preceding cycle being utilized for modifying the support-bound probes in the subsequent cycle.

1. Introduction

The detection of biologically relevant information in defined investigation material is of outstanding importance for basic research, medicine, biotechnology and other scientific disciplines. For the most part, genetic information occupies the center of attention. This genetic information comprises an enormous variety of different nucleic acid sequences, the DNA. Utilization of said information in an organism mainly leads to the synthesis of proteins via the preparation of transcripts of the DNA into RNA.

In order to be able to better understand these principles of action of nature, an efficient and reliable decoding of DNA sequences is necessary. The detection of nucleic acids and the determination of the sequence of the four bases in the chain of nucleotides, which is generally referred to as sequencing, provides valuable data for research and applied medicine. In medicine, in-vitro diagnosis (IVD) has made it possible to a greatly increasing extent to develop, and make available to the treating physician, instruments for determining important parameters of patients. Without said instruments, it would be impossible to diagnose many diseases at a sufficiently early time. Genetic analysis has become established here as an important new method.

It has been possible with close interlinkage of basic research and clinical research to trace back and elucidate the molecular causes and (pathological) relationships of some disease states down to the level of genetic information. The development of this scientific procedure is, however, still in its infancy, and much more intensive efforts are needed in particular for its realization within the framework of therapeutic strategies. Overall, the genomic sciences and the nucleic acid analytical techniques associated therewith have made important contributions both to the understanding of the molecular bases of life and to elucidating very complex disease states and pathological processes.

2. Prior art

Genetic information is obtained via the analysis of nucleic acids, mainly in the form of DNA. There are three essential techniques for the analysis of DNA, the first of which is referred to as polymerase chain reaction (PCR). This method and related methods are used for the selective, enzyme-supported duplication (amplification) of DNA by using short flanking DNA strands with known sequence, in order to start enzymic synthesis of the region located in between. In this connection, the sequence of said region may not be known in detail. On the basis of a small excerpt of information (the flanking DNA strands), said mechanism thus allows the selective duplication of a particular DNA section so that this duplicated DNA strand is available in large quantities for further studies and analyses.

The second basic technique used is electrophoresis. This is a technique for separating DNA molecules on the basis of their size. The separation is carried out in an electric field which forces the DNA molecules to migrate. Suitable media such as, for example, crosslinked gels, make movement in the electric field more difficult as a function of the size of the molecule so that small molecules and thus shorter DNA fragments migrate faster than longer ones. Electrophoresis is the most important established method for DNA sequencing and, in addition, for many processes for purifying and analyzing DNA. The most widespread method is flat-bed gel electrophoresis which, however, in the field of high throughput sequencing is increasingly superseded by capillary gel electrophoresis.

The third method is the analysis of nucleic acids by “hybridization”. For this, a DNA probe with known sequence is used in order to identify a complementary nucleic acid, most of the time against the background of a complex mixture of a large number of DNA or RNA molecules. The matching strands bind to one another in a stable and very specific manner.

The three basic techniques frequently occur in combination, for example by duplicating the sample material for a hybridization experiment selectively via PCR beforehand.

Likewise, the principle of hybridization of matching DNA strands is utilized for the sequence analysis on a DNA support chip. The development of DNA support chips or DNA arrays means an extreme parallelization and miniaturization of the formats of hybridization experiments. DNA in a sample can bind to the DNA fixed to the support only at those positions in which the sequences of the two DNA strands match. It is possible with the aid of the DNA fixed to the support to selectively detect the complementary DNA in the sample. As a result, for example, mutations in the sample material are recognized by the pattern which is produced on the support after hybridization.

When working on very complex genetic information using such a support, the main bottleneck is the access to said information via the limited number of measuring locations on the support. Such a measuring location is a reaction area in which during preparation of the support DNA molecules are synthesized as specific reaction partners, known as “probes”.

There are in principle two possibilities to achieve a higher data throughput: one possibility is to increase the number of measuring locations on a reaction support. The second possibility is to increase the number of different probes the system is able to generate per time (and per money invested) and make available for hybridization. The second possibility is somehow connected with the number of variants which are generated in the system and are made available for the analysis (data throughput).

For the term genetic information, a distinction must be made between unknown sequences which are decoded for the first time (this is generally understood by the term sequencing, also de novo sequencing) and known sequences which are to be identified for reasons other than first-time decoding. Examples of such other reasons are expression studies of genes and verification of the sequence of a DNA section of interest in an individual. This may take place, for example, in order to compare the individual sequence with a standard, as in the mutation analysis of cancer cells and the typing of HIV viruses.

For de novo sequencing, electrophoretic methods have been used almost exclusively up until now. The fastest method is capillary electrophoresis.

Supports have hardly been involved in de novo sequencing up until now. The reasons for this are some basic limitations: in order to obtain information via sequence comparison, probes need to be provided on the support. When working on unknown material, a large number of different probes (variants) are needed. None of the methods known so far is capable of generating the required numbers of variants for an effective sequencing by sequence comparison of very large amounts of DNA. Such very large amounts of DNA are present, for example, when determining the sequences of whole genomes.

Essentially two methods for preparing supports are known so far. In the first preparation method, the finished probes are prepared individually either in a synthesizer (chemically) or from isolated DNA (enzymically) and then applied in the form of tiny drops to the surface of the chip, that is each individual type of probe to an individual measuring location. The most widespread method for this is derived from the inkjet printing technique, and therefore said methods are combined under the generic term “spotting”. Likewise, methods using pins are very common. Only by micropositioning of printhead or pin is it later possible to assign a signal on the chip to a particular probe (array with rows and columns). The spotting apparatus must therefore work in a correspondingly accurate manner.

In the second method, the DNA probes are prepared directly on the chip by using site-specific chemistry (in situ synthesis). For this purpose, there are two methods available at the moment.

One of them makes use of the above-described spotting apparatuses but differs in that the tiny drops contain appropriate chemicals for the synthesis so that location-resolved chemistry can be carried out by micro-positioning of said chemicals. The technology permits random programming of the sequence of the probes being generated. However, up until now the throughput, i.e. the number of probes per time, has not really been high enough in order to convert large amounts of genetic information.

The second method, the parallel synthesis of the probes using light-dependent chemistry, makes it possible to prepare many more measuring locations per time. It has already been used to synthesize more than 100 000 measuring locations per chip in a few hours.

The method is used with two technical solutions for illumination. One of these uses photolithographic masks and generates a large number of measuring locations on the DNA support due to the highly developed optics. However, the choice of probe sequence is very limited, since appropriate masks have to be prepared. This preparation method is therefore not very suitable for the method of the invention. Methods using freely programmable probe sequences, which work on the basis of appropriately controllable light sources, are much more promising. Such preparation methods for probes on a support are described, inter alia, in the patent applications DE 198 39 254.0, DE 198 39 256.7, DE 199 07 080.6, DE 199 24 327.1, DE 199 40 749.5, PCT/EP99/06316 and PCT/EP99/06317.

In summary, it can be stated that the previously established techniques for processing relatively large amounts of genetic information with completely or partially unknown composition, namely electrophoretic methods and biochip supports, limit the throughput. Up until now, high-throughput projects for de novo sequencing have relied on sorting by size using electrophoresis (inter alia, the Human Genome Project HUGO). Due to miniaturization and parallelization, improvements can be expected here but no breakthroughs, since the technique per se cannot be altered. Electrophoresis can manage most applications of biochips, such as, for example, expression patterns or screening for mutations, only much more slowly, if at all. The biochips known up until now are for their part unsuitable for de novo sequencing, and the focus is on highly parallel processing of material on the basis of known sequences (inter alia in the form of synthetic oligonucleotides as probes).

Both formats have a limited throughput of genetic information. In order to increase this throughput, new approaches have to be developed. The method of the invention is such an approach.

3. Subject of the Invention

The invention relates to a method for sequencing nucleic acids, which comprises the following steps:

(a) carrying out a first hybridization cycle, comprising

(i) providing a support having a surface which contains immobilized hybridization probes on a multiplicity of predetermined areas, said hybridization probes in individual areas having in each case a different base sequence of a predetermined length,

(ii) contacting a sample which contains nucleic acids to be sequenced with the support under conditions under which a hybridization between the nucleic acids to be sequenced and probes complementary thereto on the support can take place, and

(iii) identifying the predetermined areas on the support, in which a hybridization has taken place in step (ii),

(b) carrying out a subsequent hybridization cycle, comprising:

(i) providing a further support having a surface which contains immobilized hybridization probes on a multiplicity of predetermined areas, said hybridization probes in individual areas having in each case a different base sequence of a predetermined length, for said further support hybridization probes having a base sequence being selected for which in the preceding cycle a hybridization has been observed, and the selected hybridization probes being extended by at least one nucleotide compared with a preceding cycle,

(ii) repeating step (a) (i) using the further support, and

(iii) repeating step (a) (iii) using the further support, and

(c) carrying out, where appropriate, further subsequent hybridization cycles in each case with selection and extension of the hybridization probes according to step (b) (i), until there is sufficient information about the nucleic acids to be sequenced.

The method described here for sequencing nucleic acids by hybridization allows, with the aid of iterative dynamic construction of all specific probes required therefor, the sequencing of sample material (even much larger than 10 kbp) of unknown sequence. The sequencing comprises both an analysis of fragments (a few dozen to 100 bp) and the mapping of said fragments within the starting sequence.

In this connection, support or reaction support is intended to mean both open and closed supports. Open supports may be planar (e.g. cover slip) or else may have a specific shape (e.g. dish-shaped). For all open supports, the surface means an area on the outside of the support. Closed supports have an inside structure which comprises, for example, microchannels, reaction spaces or/and capillaries. Here, surfaces of the support mean the surfaces of the two- or three-dimensional microstructure inside the support. The combination of closed surfaces on the inside and open surfaces on the outside of a single support is also possible, of course. Examples of materials used for supports are for example glass such as Pyrax, Ubk7, B270, Foturan, silicon and silicon derivatives, plastic materials such as PVC, COC or Teflon and Kalrez.

The array required for the method need not necessarily be limited to one support and it is perfectly possible to distribute a “virtual array” on several supports. This makes it possible to increase the number of positions, if necessary.

In a closed system which may contain both sample preparation and fragmentation and mapping of the sample material, see, for example, DE 199 24 327.1, DE 199 40 749.5 and PCT/EP99/06317, data generation and evaluation mutually complement and depend on each other and together form a learning unit. Thus, for example, new probe sequences are determined with the aid of the evaluated data of one array, which sequences are then synthesized on a new array. This is carried out systematically until the biological variety which represents only a very small part of the theoretically possible variations has gradually been captured in its entirety.

In the method of the invention, probes are prepared on or in the support in a flexible manner so that a flow of information becomes possible. Each new synthesis of the array in successive cycles can take into account the results of a previous experiment. Choosing the hybridization probes which may be oligonucleotides or else nucleic acid analogs such as peptidic nucleic acids in a manner appropriate with respect to their length, sequence and distribution on the reaction support and a feedback of the system via integrated signal evaluation make it possible to process genetic information efficiently.

The invention furthermore relates to a support for sequencing nucleic acids, which has a surface containing immobilized hybridization probes in a multiplicity of predetermined areas, said hybridization probes in individual areas having in each case a different base sequence of a predetermined length, it being possible for said hybridization probes to have, in addition to variable sections, one or more sections that are fixed for at least part of the probes.

The method and the support may be employed for determining the sequences of genomes, chromosomes, transcriptomes and for identifying polymorphisms in nucleic acid sequences, for example at the level of single individuals.

Binding of the nucleic acids to hybridization probes in the particular sections on the support surface is preferably detected via marker groups. The marker groups may be bound directly or indirectly to the nucleic acids to be sequenced. Preference is given to using marker groups which are optically detectable, for example by fluorescence, refraction, luminescence or absorbance. Preferred examples of marker groups are fluorescent groups or optically detectable metal particles, for example gold particles.

### 4. DETAILED DESCRIPTION OF THE INVENTION

4.1 (Numerical) Relations

At the beginning, a few relationships which play an important part hereinbelow will be explained:

In each sequence consisting of m nucleotides no more than m−n+1 part sequences of length n may be present. This means that for each total sequence length m there is a specific sequence length n for which the number of all possible n-mers (4ⁿ) exceeds the number m−n+1 of the part sequences of length n possible within the total sequence. Thus, for example, no more than approx. 3.2×10⁹ sequence sections of a random length n may be present in the human genome which consists of approx. 3.2×10⁹ nucleotides. If n=16, then the number of all 16-mers, 4¹⁶, is distinctly greater than the maximum number of 16-mers present in the human genome. Thus it is impossible in any case for all 16-mers and thus also for all longer (n+1)-, (n+2)-mers, etc. to be present in the human genome.

Table 1 shows the relationship between sequence section length n, sequence length m and the maximum number of part sequences of length n contained in the sequence of length m. In each sequence shorter than the value given for m it is not possible for all possible sections of a given length n to be present.

Considering all n-mers present in a sequence of length m, which follow a part sequence of length p, the number of said n-mers is distinctly smaller than the above-described number of m−n+1 part sequences.

A sequence containing all, 4^{P}, possible p-mers must have a minimum length of k=4^{P}+p1 nucleotides. Assuming that all p-mers occur with the same probability, then each p-mer is present in a sequence of adequately chosen length on average every k nucleotides; thus, in a sequence of length m with m>>k, l=m/k=m/4^{P}=p−1 times. Consequently, it is possible to detect in such a sequence of length m also no more than 1 n-mers which follow a p-mer.

TABLE 1

| | Sequence length | n-mers in sequence |
|---|---|---|
| n | m | m − 4\hat{ }n + 1 |
| 3 | 66 | 64 |
| 5 | 1028 | 1024 |
| 6 | 4101 | 4096 |
| 7 | 16390 | 16384 |
| 8 | 65543 | 65536 |
| 9 | 262152 | 262144 |
| 10 | 1048585 | 1048576 |
| 12 | 16777227 | 16777216 |
| 13 | 67108876 | 67108864 |
| 14 | 268435469 | 268435456 |
| 15 | 1073741838 | 1073741824 |
| 16 | 4294967311 | 4294967296 |
| 17 | 17179869200 | 17179869184 |
| 18 | 68719476753 | 68719476736 |
| 19 | 2.74878E+11 | 2.74878E+11 |
| 20 | 1.09951E+12 | 1.09951E+12 |
| 25 | 1.1259E+15 | 1.1259E+15 |

If, for example, in the human genome (single-stranded) a randomly but permanently chosen 3-mer is chosen and all sequence sections of length n following said 3-mer are investigated, no more than 48 500 000 different n-mers are found, assuming equal distribution of all p-mers.

In this case, too, there is a characteristic limit for the variety of part sequences. If the chosen studied part sequences are longer than the length n corresponding to the maximum variety, then there are more possible variants than can occur in the sequence investigated. In the human genome (with all generalizing assumptions) this is a sequence length n=13; there is a total of 4¹³=67108864 sequences of length 13. However, in the human genome there can be, as calculated above, only approx. 50 000 000 different part sequences downstream of a freely chosen 3-mer. For any longer part sequence length, it is in any case impossible for all possible variants to occur in the genome.

Table 2 shows with the aid of a few examples the relationship between the sequence length m, the choice of p and the length n of the part sequence which is to be studied downstream of the p-mer. The third column lists the, with idealized assumptions, average occurrence of the chosen p-mer in the starting sequence; from this, the value for n for which the complete variety of n-mers downstream of the p-mer can still occur, is determined. For any greater p chosen or for any shorter sequence chosen this is no longer true.

A longer p-mer restricts the variety within the sequence investigated considerably more than a shorter p-mer, since the longer p-mer is relatively rarer.

TABLE 2

| Sequence | | Occurrence | | Number of |
|---|---|---|---|---|
| length | | of p-mer | | n-mers |
| m | p | m/(4\hat{ }p + p − 1) | n | 4\hat{ }n |
| 4352 | 2 | 256 | 4 | 256 |
| 16896 | 3 | 256 | 4 | 256 |
| 66304 | 4 | 256 | 4 | 256 |
| 17408 | 2 | 1024 | 5 | 1024 |
| 67584 | 3 | 1024 | 5 | 1024 |
| 265216 | 4 | 1024 | 5 | 1024 |
| 17825792 | 2 | 1048576 | 10 | 1048576 |
| 69206016 | 3 | 1048576 | 10 | 1048576 |
| 271581184 | 4 | 1048576 | 10 | 1048576 |
| 285212672 | 2 | 16777216 | 12 | 16777216 |
| 1107296256 | 3 | 16777216 | 12 | 16777216 |
| 4345298944 | 4 | 16777216 | 12 | 16777216 |
| 1140850688 | 2 | 67108864 | 13 | 67108864 |
| 4429185024 | 3 | 67108864 | 13 | 67108864 |
| 17381195776 | 4 | 67108864 | 13 | 67108864 |
| 4563402752 | 2 | 268435456 | 14 | 268435456 |
| 17716740096 | 3 | 268435456 | 14 | 268435456 |
| 69524783104 | 4 | 268435456 | 14 | 268435456 |

The method described below utilizes this reduction in variety. Thus it is, for example according to the considerations above, not necessary to synthesize the total amount of all 25-mers on one array, in order to make a statement about which 25-mers occur in a sample sequence. Depending on the length of the sequence investigated, only a very small proportion of all 25-mers may occur in said sequence, see Table 1.

4.2 Dynamic Array Construction

Compared with the previously common (static) methods of generating support chips, it is possible according to the invention to learn quickly from one array to the subsequent array and thereby obtain many times the previous amount of information.

If, over a short period, different arrays can be generated using the information obtained after evaluating the preceding array, the system becomes a “learning” system. Using this method it is possible to determine the abovementioned 25-mers of a sequence without having to synthesize them in their diversity (4²⁵=1.125899907×10¹⁵).

It is possible, for example, to start with a variable probe length s under which the possible diversity (4^{s}) of all s-mers can be synthesized on the array. If all possible 4^{S} sequence variations cannot be generated on a single support, it is possible also to use a limited number of several supports for one hybridization cycle. If the length of the probes is below the value n determined in Table 1, it is possible, but not probable, that all sequences generated on the array occur in the starting sequence. In addition, this probability decreases with increasing length of the probes. In any case, however, not more than the part sequences calculated in Table 1 can occur in the sequence.

In the next step, all probes which have generated a signal on the preceding array are synthesized on a new array and extended in each case by at least one nucleotide in all possible variations, i.e. an extension by one nucleotide leads to four hybridization probes extended in different ways. At the latest from the part sequence length n listed in Table 1 onward, the number of signals will not increase further, since their number (with idealized assumptions) cannot be greater than the maximum number of the different part sequences in the starting sequence. With “normal” assumptions, there will be signals which should not have been produced according to idealized assumptions. These probes may initially be constructed further and possible errors in the course of the iteration may be eliminated by extended probes and the more specific bonds resulting therefrom. In addition, in practice the complete diversity of all possible part sequences will never be present in a sequence to be investigated so that distinctly fewer signals than the maximally possible number are generated.

Depending on the number of positions and the length of the sequence to be investigated, preference is given to choosing the probe length of the first array such that, after hybridization, no more than 25% of all positions emit signals. This procedure ensures that the number of probes does not increase in the next step. Thus the length chosen for the probes on the new array may be one base greater than the length of the probes on the preceding array without an increase in the number of probes.

To choose the starting probes in this way, the length m of the sequence (in this case a single strand, similar rules apply for a double strand) must be smaller than the permitted number of signals, in formulae: m≦4^{s-1}+s−1, where s is the probe length. Thus, on an array with probe length s=6, a sequence of the maximum length m=4⁵+5=1029 can be processed so that, after hybridization, at any rate less or no more than 25% of all probes emit signals. The following Table 3 shows the preferred length s of the starting probes as a function of the length m of the sequence to be determined.

TABLE 3

| Probe length s | Sequence length m |
|---|---|
| 5 | 260 |
| 6 | 1029 |
| 7 | 4102 |
| 8 | 16391 |
| 9 | 65544 |
| 10 | 262153 |
| 11 | 1048586 |
| 12 | 4194315 |
| 13 | 16777228 |
| 14 | 67108877 |
| 15 | 268435470 |
| 16 | 1073741839 |
| 17 | 4294967312 |
| 20 | 2.74878E+11 |
| 22 | 4.39805E+12 |
| 25 | 2.81475E+14 |

Since in a sequence of length m part sequences of length s can quite possibly appear several times, the theoretical number of m−s+1 part sequences of length s is often reduced in practice. In that case, a smaller probe length is sufficient. However, since the number of repetitive sequences is not known at the start, the value determined above must be regarded as an upper limit. The repeated appearance of a part sequence reduces, but never increases the number of signals.

Some numerical examples:

For the human genome which has 3.2×10⁹ nucleotides per strand, a probe length of 17 bases is sufficient in order to ensure theoretically that binding on the array takes place at less than 25% of all positions. For E. coli which has 4 639 221 nucleotides, even probes of length 13 are sufficient. The number of positions on all subsequent arrays will not exceed the number of positions on these arrays.

If the length of the probes on the first array is not chosen according to the above-described method, the number of signals will in any case settle down during the course of the process below the maximum value of m−n+1, where n is the length described in the first section, for which the diversity of all n-mers is greater than the number of n-mers possible in the starting sequence. If the probe length chosen at the start is too short, the number of required positions will in the next steps first increase up to a maximum of 4ⁿ⁻¹ positions and then stagnate. If the probes chosen are too long, distinctly fewer than 25% of all positions will be successful during hybridization so that the number of required positions in the next step is automatically reduced.

As described in the first section, the diversity of the part sequences in a sequence of length m can be reduced still further by considering only sequence sections which follow a previously determined sequence of nucleotides. In this case, too, the length of the probes on the first array can be determined as above. This means for an array on which all combinations of length s=n+p which begin or end with the p-mer are synthesized, that signals must be met only from no more than 25% (i.e. l/4ⁿ%) of all 4ⁿ⁻¹ positions on this array. Thus it is possible to hybridize on an array with probe length s=n+p and a random section of length p, which is, however, fixed for all or part of the probes, a sequence of length m, with m≦4ⁿ⁻¹×(4^{p}+p−1), without the theoretically possible number of positions which can emit signals exceeding 25% of the total number of positions; n in this connection is the value calculated in the first section.

Table 4 illustrates for some examples the relationship between the maximum length of the starting sequence and the length of the probe and of the p-mers. With a fixed 3-mer, a probe length of n+p=17 nucleotides is sufficient for the human genome in order not to exceed the permitted number of positions delivering a signal. The number of probes to be synthesized is in any case 4ⁿ, i.e. the number of all possibilities of constructing the flexible part of the probes.

The values calculated above and in the first section are valid for an equal distribution of the p-mers studied. For most sequences, this idealized assumption is not true and greatly different distributions of the individual nucleotides may be present. If therefore, for example, in the case of DNA/RNA sequences the A-T or C-G content of the sequence to be investigated is known, it is possible to calculate probabilities for the individual p-mers. When calculating the maximum sequence length, a weighting with the aid of the probability for the presence of the chosen p-mer will in some cases cause a shift in the values listed in Tables 2 and 4.

Table 4: Greatest possible length of the starting sequence in relation to the probe length and to its composition

| | | Probe length | Sequence length |
|---|---|---|---|
| n | p | s = p + n | m |
| 4 | 3 | 7 | 4224 |
| 4 | 4 | 8 | 16576 |
| 5 | 3 | 8 | 16896 |
| 5 | 4 | 9 | 66304 |
| 8 | 3 | 11 | 1081344 |
| 8 | 4 | 12 | 4243456 |
| 10 | 3 | 13 | 17301504 |
| 10 | 4 | 14 | 67895296 |
| 12 | 3 | 15 | 276824064 |
| 12 | 4 | 16 | 1086324736 |
| 14 | 3 | 17 | 4429185024 |
| 14 | 4 | 18 | 17381195776 |
| 15 | 3 | 18 | 17716740096 |

The dynamic construction of a sequence of arrays has the advantage that, after evaluating the information of the preceding array(s) a new array can be constructed which provides the required data. It is possible to obtain information about part sequences in the starting sequence of a specific length, for example of 25 bases and more, without having to construct all possible combinations of this length. The method automatically reaches a maximum signal number and thus a maximum number of positions per array.

An application which can be realized using the above-described dynamic array construction is described below.

4.3 Dynamic Sequencing by Hybridization (DSBH)

At this point, first the general principle of DSBH is described which is made possible essentially by a flexible construction of the arrays; possible realizations of this principle follow in the next section.

As described above, p-mers occur in a sequence to be determined with different probabilities which can be determined, for example, in the case of DNA sequences by knowing the A-T and G-C content of the sequence. The basic idea of DSBH is to select p-mers which occur in the sequence at regular intervals; they can be thought of as “islands” whose sequence is already known. Starting from these fixed points of known sequence (POKS), the sample sequence is then determined. For this purpose, initially three types of probe are required on the arrays:

(1) probes with fixed sequences at the 3′ end,

(2) probes with fixed sequences at the 5′ end,

(3) probes with fixed sequences inside, for example in the center of the sequence.

The probes (1), (2) and (3) may be employed together or/and successively on the same support or on different supports. For the first two types of probe, all combinations of a predetermined length are synthesized, the complementary sequence to the chosen POKS being synthesized once at the 3′ end of the sequence and once at the 5′ end of the sequence. The hybridization of the starting sequence against the probes of this array then provides information about all nucleotide combinations of the predetermined length, both in 3′-5′ direction toward the POKS and in 3′-5′ direction away from the POKS. After the above-described procedure of dynamic construction of the arrays, all probes of the positions which have generated a signal are synthesized on a new array and in each case extended by one nucleotide in all four variations. With a sufficiently large number of positions on the array, it is also possible to process two or more iteration steps on one array, i.e. an extension by two or more nucleotides may be carried out.

When extending the probes, it must be taken into account that probes for which the POKS-complementary sequence is synthesized at the 3′ end are extended in 5′ direction and probes with the complementary POKS sequence at the 5′ end correspondingly in 3′ direction. Once the iteration has reached a maximum probe length, the sequence of the nucleotides on both sides of each POKS is known for the length of the maximum probe length. In this connection, the probe length is limited either by the possibilities of the system used or by a compromise between the time required until the final result and the accuracy thereof.

With the aid of the third type of probe, the sequences determined above are connected. Now all those probe sequences whose POKS-complementary sequence is in the center and which has upstream or downstream of said complementary sequence parts of the information obtained by the first two probes are determined. These probes are synthesized on a new array; after hybridization and evaluation of the signals, all possibilities of permitted combinations of the sequences determined by the first two types of probe are known.

This information may likewise be obtained by an iterative array construction in which all combinations of a particular length upstream and downstream of the POKS-complementary sequence are synthesized. After evaluation of the signals, the relevant probes are extended further as described above, now in both directions, etc. However, with an adequate number of positions it is possible to avoid these iteration steps by constructing the required probes immediately up to the maximum length.

The array with the third type of probe solves in a highly parallel manner a combinatorial problem which, without a flexible array construction, can be solved only with very great computational effort with the aid of computers. Shifting this task to the array means considerable time savings compared with a combinatorial computer calculation and moreover provides more reliable data.

If the POKS are then chosen appropriately, it is possible to reassemble the starting sequence using the above-described method by comparing and combining the overlapping sequences of the part sequences determined via the individual POKS.

The following sections 5 and 6 illustrate in detail two particularly preferred embodiments of the method of the invention.

5. Dynamic Sequencing by Hybridization (DSBH) Using Statistically Chosen Fixed Probe Sections (POKS)

5.1 Prerequisites

The method for sequencing using POKS which have been chosen statistically or via said method and also the corresponding sample preparation are described for a single strand. It is also possible to sequence double-stranded nucleic acids using the same method.

5.1.1 Sample Preparation

The sequencing described here starts from single-stranded nucleic acids. In the simplest case, these may be isolated directly in the form of single-stranded RNA or DNA from viruses, bacteria, plants, animals or humans. In the majority of cases, however, the single-stranded nucleic acids are generated by specific in vitro methods starting from dsDNA. Said specific methods include, for example, asymmetric PCR (generates ssDNA), PCR with derivatized primers which enable selective hydrolysis of an individual strand in the PCR product, or transcription by RNA polymerases (generates ssRNA). The template used for transcription may be, in addition to uncloned single-stranded DNA, especially also dsDNA cloned into specific vectors (e.g. plasmid vectors with one promoter; plasmid vectors with two promoters with different orientation for a particular RNA polymerase or two different RNA polymerases). The DNA insert cloned into the plasmids or the DNA template used in the PCR may on the one hand be isolated from viruses, bacteria, plants, animals or humans or else on the other hand be generated from ssRNA in vitro by reverse transcription, RNaseH treatment and subsequent amplification (e.g. by PCR). RNA templates which may be used are rRNAs, tRNAs, mRNAs and snRNAs and also transcripts generated in vitro (produced, for example, by transcription with SP6, T3 or T7 RNA polymerase).

The single-stranded nucleic acids provided for sequencing are fragmented sequence-specifically or/and sequence-unspecifically (e.g. by sequence-(un)specific enzymes, ultrasound or shear forces), striving for an essentially homogeneous length distribution of the fragments/hydrolytic products. If no homogeneous distribution of the fragment length is obtained, it is possible to carry out subsequently a fractionation by length using gel-electrophoretic and/or chromatographic methods.

The fragments produced may be labeled by marker groups, for example fluorescent agents or radioactive isotopes. The labeling is preferably carried out on the fragment ends (terminal labeling). 3′-terminal labeling reactions may be carried out using suitable synthons, for example with terminal transferase or T4 RNA ligase. If in vitro-generated RNA transcripts are used for the fragmentation, the labeling may also be carried out prior to the fragmentation via labeled nucleotides used in the transcription (internal labeling).

The labeled fragmented nucleic acids may then be hybridized against the support coated with a probe array in a suitable hybridization solution.

5.2 Selection of the Fixed Probe Sections (POKS)

In the following variant of the method for sequencing using POKS, p-mers selected according to different criteria serve as POKS; they may be determined at different points in time during the process.

Firstly, it is possible to determine a fixed number of POKS at the start of the process. It suggests itself here to select those combinations (p-mers) which occur with the highest probability in the starting sequence. This is possible because the individual nucleotides and thus also the individual p-mers occur with different probabilities in the sample sequence, as described in the first section. If, for example in the case of DNA sequences, the G-C content and A-T content of said sequence are known, it is then possible to determine those p-mers which are most probably, and thus most frequently, present in the sequence. Other methods for choosing the POKS at the start of the process, for example from empirical data or by random determination, are likewise possible.

Secondly, it may be sensible to determine only a few or one POKS at the start of the process and all subsequent POKS from the sequence information obtained thus far. This strategy enables the method to learn from the previously generated data and to determine which data are important for the further course of the process and for combining the information. The first POKS need not necessarily be predetermined by the user and may, for example as illustrated above, be determined by the system via determination of the probabilities for the potential POKS, from empirical data or randomly.

When choosing the POKS at the beginning of the process, first the number of POKS must be determined. These may be determined, for example, from empirical data or be calculated statistically by choosing such a large number that the distance between two POKS is for purely mathematically reasons distinctly shorter than the predetermined maximum probe length on the arrays.

If the POKS are determined only during the course of the process then the number can either be determined beforehand, see above, so that the process is stopped when the maximum POKS number is reached, or further POKS are determined until other stopping criteria are met. It is, for example, possible to stop the process if a sequence of a predetermined length has been assembled, which meets all demands on a potential solution of the problem. Likewise, the process may be stopped, for example, when the previously assembled sequences can no longer be extended at either end.

5.3 Procedure

The method is based essentially on the above-described dynamic array construction, since said construction allows sequence information of a specific length to be obtained without having to generate for this purpose the probes in all their diversity. In addition, the parallel “computing power” of the arrays is utilized, which renders time-consuming and complex processes unnecessary.

5.3.1 Various Types of Probe on the Array

For all initially determined POKS, the three above-described types of probe are synthesized on one or more arrays, i.e. all combinations of a predetermined length are generated once on the 3′ end using the POKS-complementary sequence and once on the 5′ end using said sequence. Hybridization with the starting sequence provides, after the signal analysis, information in the form of (approximate) probe length about the pairs of nucleotides to the right and the left of said POKS. It is possible to generate iteratively new probes with the aid of said signals, as described above. This is repeated until a maximum probe length is attained. At this point, all possible combinations on maximum probe length at both sides of each POKS in the starting sequence are known.

TABLE 5

| | | | |
|---|---|---|---|
| N | P | N | 5′ end |
| N | P | N | |
| N | P | N | |
| N | N | N | |
| N | N | N | |
| N | N | N | |
| N | N | P | |
| N | N | P | |
| N | N | P | |
| N | N | N | |
| N | N | N | |
| N | N | N | |
| P | N | N | |
| P | N | N | |
| P | N | N | 3′ end |

Table 5 shows the three different types of probe with the POKS (PPP) or the complementary sequence thereof at the 3′ end, at the 5′ end and inside the probe.

With the aid of the third type of probe, the connection between said pieces of information is established. The center of each probe contains the sequence complementary to the chosen POKS and, on both sides of said sequence, all possible combinations of a particular length are generated in various probes. Using the same iterative procedure as for the first two types of probe produces information about all combinations of the sequences present in the starting sequence, which have been detected up until then. If the number of positions required for the third type of probe, which is derived from the number of all possible combinations of the detected sequences, is lower than the number of positions on the array, the parts of the detected probes of the first and second types can be incorporated directly into the new probes. In this case, an iteration is not required. The direct generation of all possible connections between the detected sequences requires distinctly fewer positions.

5.3.2 Assembly of the First Pieces of Sequence Information

After analysis of the arrays using probes of the third type and an intermediate computer step, all combinations of length

K=2×maximum probe length−length of POKS,

which can be present in the starting sequence, are known; they all have a POKS in the center of the sequence.

These part sequences can then be extended with the aid of the POKS. For this purpose, each part sequence is searched on one or both sides of the central POKS for a new site at which one of the POKS used is present. If a POKS is found, the sequence information on both sides of said POKS is compared with all part sequences which contain exactly this POKS. This procedure makes it possible to link the individual part sequences and a tree of all variants into which said sequences can be combined is produced.

Table 6 below shows two overlapping part sequences in a DNA sequence which has been identified with the aid of a POKS.

TABLE 6

ATGGAGCACTTGGPPPCCTACGPPPGTCA
TTGGPPPCCTACGPPPGTCATTGGCAGTA

In the upper sequence of Table 6, another POKS was found at position 7 to the right of the central POKS. Comparison with the second sequence which contains the “identified” POKS in the center of the sequence has found that the two sequences overlap to the greatest possible extent, namely from position one of the second sequence to position 20 of this sequence.

If all POKS have already been determined at the start of the process, then all possible neighborhood connections of the part sequences are known. The nucleotide combinations may be combined to the total sequence, and for this purpose the tree of all possible combinations is screened and part sequences which appear to make sense are combined to a total sequence. If repetitive part sequences appear, the algorithm is stopped after a few cycles; in this connection, a possible stopping criterion is, for example, the assumed length of the starting sequence.

Finally, all potential solution sequences have to be checked to see whether they are correct, in order for the error between the solution sequence determined and the starting sequence to be as small as possible.

5.3.3 Determination of New POKS

If not all of the POKS have been predetermined immediately at the start of the process, it is now possible to determine new POKS from the already known sequence parts. To this end, there are several variants. First, it is possible to investigate all part sequences on one side of the POKS in the center of each sequence for the p-mers appearing most frequently, where p is the length of the POKS to be chosen, which can be either predetermined or optimized during the process. This choice of POKS makes it-possible in the next step to determine a sequence for a majority or for all part sequences known up until then, by which sequence the previously detected sequences can be extended. In order to ensure that a subsequent sequence or a preceding sequence is found for each part sequence, a relatively large number of POKS may be required. The newly determined POKS are used for generating the same probes as have been generated using the POKS chosen at the start. The information obtained thereby provides new possibilities of assembling and extending the known part sequences. If the criteria for stopping the process are not yet met, POKS are again determined from the newly determined sequences and used for obtaining new information.

In order to reduce the number of POKS required, it is sensible first to assemble the information obtained using the POKS chosen at the start of the process into longer sequences. These longer sequences are, if required, compared to each other and shorter sequences which can also be found in the longer sequences are deleted. All of the remaining sequences end in part sequences for which no subsequent sequence can be determined or start with sequences for which there is no preceding sequence. In these “end sequences” frequently occurring p-mers are then determined as above. The p-mers serve as new POKS for which again the three types of probe are generated and for which thus, after signal analysis, all possible base combinations around the POKS are known.

Only in the start sequence and the end sequence of the sequence to be investigated it is possible to find POKS without said sequences being extended further. If these part sequences are identified in the process, they are treated separately and are not included in the determination of new POKS.

Owing to the choice of the new POKS, the newly determined sequences then overlap partly with the already known longer sequences which are then, if possible, extended in both directions. Moreover, all those combinations are generated, which form due to the new POKS and are not yet included in the already known sequences. New POKS are again generated from the new “end sequences”; this takes place until one of the stopping criteria is met.

Apart from the methods listed above for determining the POKS, other procedures in which POKS are determined after the individual process steps are also conceivable, of course. Inter alia, a combination of various methods may prove suitable.

By choosing the new POKS on its own, the system develops a learning process in which there is an interdependence between the evaluation of data and the composition of new arrays for obtaining new data.

5.3.4 Final Assembly and Verification of the Sequences

If the POKS are determined at the start of the process, the identified part sequences are assembled into long sequences in all possible combinations. When selecting the POKS appropriately, each part sequence overlaps with another one so that the original sequence is among the combined possibilities. In order to find out which of the sequences is best suited to solve the problem, all sequences are checked first among each other for overlaps. If such overlaps are present and if a sequence assembled from the overlapping part sequences does not exceed the estimated or known length of the sample sequence, then the sequences are combined further. Short sequences which are completely contained in longer sequences are deleted.

Apart from the sequence length, comparison with all part sequences detected on the arrays provides a clue to determining the sequence which is the best match for the sample sequence. Ideally, the solution sequence contains all, or at least a large part, of the sequences determined on the arrays by the first two types of probe; under no circumstances can base combinations which have not been identified on the arrays be present upstream or downstream of a POKS.

If, in addition, the signals obtained can be quantified, i.e. if it is possible to determine, at least approximately, how often a detected sequence occurs in the original sequence, then this is another criterion during verification. A sequence must not occur more frequently than as identified.

Besides the criteria listed above, it is of course possible to investigate, as a control, the same sequence using other POKS and to compare the results, a process which can perfectly run in parallel with a high density of positions on the arrays.

If the POKS are determined only during the course of the process, it is possible to detect already in each step whether the individual sequences contain only part sequences which also occur in the sample sequence or whether sequences are present which are not allowed to be and thus a sequence is eliminated as solution sequence. Likewise, it is possible (during the abovementioned quantification of the signals) to ensure already after each step that a part sequence is incorporated only as often as is permitted.

5.3.5 Stopping Criteria

With a predetermined number of POKS, it is possible to stop the process automatically, if said number is exceeded after or during determination of new POKS or if, in the case of predetermined POKS, all information obtained thereby has been processed.

If both the POKS and the number thereof can be chosen freely, another stopping criterion must be found. First, of course, determination of p-mers is limited by the number thereof, since there are exactly 4^{p} p-mers. Depending on the choice of p, this number is relatively high and thus too large to serve as a natural stopping criterion.

Without any previous knowledge about the nature of the sequence to be investigated (e.g. without knowing its length) the process may be stopped, if a subsequent sequence or a preceding sequence has been found for each theoretically extendible identified part sequence. At this point in time, the complete sequence information of the starting sequence is present so that it is not possible to obtain any new information from renewed determination of POKS.

If the length of the sequence to be investigated is known, the cyclic POKS determination can be ended as soon as a sequence has been found whose length matches the approximate starting length and which contains (almost) all part sequences identified on the arrays.

Moreover, it is possible to determine for the assembled sequences during the process probabilities for their “correctness” or values for estimating errors so that the process can be stopped as soon as the error falls below a previously set threshold.

5.3.6 Repeats Within the Starting Sequence and Repetitive Sequences

If repeats are present in the sample sequence, a closed circle may form in the above-described tree of all possible sequence combinations, which circle makes assembling of the sequences more difficult.

In this connection, the length of the repetitive sequence sections is of crucial importance. Repeats which are shorter than the maximum probe length (when using all 3 types of probe) or shorter than the halfmaximum probe length when using exclusively the third type of probe do not cause any problems during assembling. If repeats which are longer than the above-described ones but shorter than the total length of the part sequences minus the length of the POKS are present, then it is possible to solve said repeats by cleverly moving the POKS, i.e. by choosing a new POKS which is located very close to the POKS in the center of the sequence. If longer repeats are present, the algorithm for assembling is stopped when they appear, thus resulting in a plurality of part sequences of different length, which in each case overlap by the length of the repeats. The use of other methods such as, for example, PCR or the choice of new types of probe makes it possible to establish the connection between said part sequences.

Another possible approach for solving the phenomenons caused by repeats is the knowledge about the approximate length of the starting sequence. If this length is considerably exceeded when trying to assemble the identified part sequences, then part sequences have probably been incorporated too frequently. Such a sequence cannot be permitted as a result of the process.

If, in addition, it is possible to determine an order of magnitude for the frequency of the appearance of each probe in the starting sequence by quantifying the signals obtained after the hybridization, the length of the starting sequence is not necessarily required as a stopping criterion.

In the case of repetitive parts, i.e. uninterrupted repeats of relatively short sequences, appearing in the sample sequence, the possible quantification of the signals on the arrays also facilitates assembling of the sequence.

5.4 Sequencing Using Long Probes

If it is possible to choose sufficiently long probe lengths in the above-described method, the construction of the first two types of probe for each POKS can be dispensed with. It is then possible to choose the length of the probes such that the probability of another POKS in their sequence is high enough in order to guarantee overlaps. As described above, all combinations of a predetermined length are then generated for the now exclusively relevant third type of probe which contains the complementary sequence of the chosen POKS in the center of the sequence; this is followed by a hybridization against said combinations and signal-providing probes are further synthesized in the next step. In this connection, it is possible to extend each probe equally in both directions away from the POKS or alternately in one and then in the other direction until the maximum possible length is attained. Depending on the number of positions, it is again possible to process a plurality of iteration steps on one array.

The use of long probes eventually renders the construction of the first two types of probe unnecessary. This means a reduction in the positions and thus in the arrays required. On the other hand, possible errors which are produced by the calculated extension of the probes of the third type with the aid of the probes of the first and second types can be ruled out.

6. Dynamic Sequencing by Hybridization (DSBH) with Fixed Sections (POKS) Chosen via Enzyme Recognition Sites

Another variant of the method is to integrate the POKS already in the sample preparation by cutting the sample material into appropriate fragments by means of sequence-specific nucleases. The bases forming the nuclease recognition sequences then automatically serve as POKS.

6.1.1 Sample Preparation

The sample preparation for this variant of the method initially starts from dsDNA. This dsDNA may firstly be isolated as genomic, chromosomal DNA, as an extrachromosomal element (e.g. as plasmid) or as a component of cell organelles from viruses, bacteria, animals, plants or humans, but secondly may in principle also be generated from ssRNA in vitro by reverse transcription, RNaseH treatment and subsequent amplification (e.g. by PCR). RNA templates which may be employed are, in addition to rRNAs, tRNAs, mRNAs and snRNAs, also in vitro-generated transcripts (produced, for example, by transcription using SP6, T3 or T7 RNA polymerase).

The isolated or in vitro-synthesized dsDNA is then hydrolyzed using a restriction endonuclease or a mixture of a plurality of restriction endonucleases, resulting in double-stranded subfragments with defined starting and/or end sequences. The number and length of the resulting subfragments can be controlled by selecting suitable enzymes (these may also be enzymes modified or generated by protein design). For fractionation by length, the hydrolysis may be followed by gel-electrophoretic and/or chromatographic separation processes. RNA subfragments may be generated by using ribozymes.

The subfragments generated are preferably labeled after fractionation. Although labeling is in principle also possible prior to denaturation (for example by filling in 3′-cohesive ends using a DNA polymerase), the subfragments are preferably labeled after denaturation, i.e. at the level of single-stranded subfragments. The labeling is preferably carried out by means of fluorescent agents (e.g. fluorescein or Cy5), but other labeling methods such as, for example, incorporation of radioactive isotopes are also possible. The labeling groups are coupled to the subfragments mainly in the form of labeled nucleotide derivatives. Coupling at the 3′ terminus may be carried out, for example, by T4 RNA ligase or by terminal transferase (using appropriate nucleotide derivatives).

The labeled single-stranded subfragments may then be hybridized in a suitable hybridization solution against the support coated with a probe array.

6.2 Process Sequence

The sample prepared in a suitable manner is split into very small subfragments by a cutting enzyme. The sequence complementary to the nucleotide sequence of the cutting enzyme here forms directly the POKS sequence, meaning that the possible POKS are predetermined by the available enzymes. The statistical behavior of fragment length and number is analogous to that of the freely chosen POKS, due to the starting sequence and the cutting sequence used.

The thus enzymically cut up sample is sorted, i.e. fractionated, according to the length of the subfragments. Labeled subfragments which are not longer than the maximum probe length are applied to the array for analysis, according to the method described. Those probes which have found a hybridization partner among the subfragments in the sample on the first array are accordingly extended cyclically to the maximum probe length. This results in determining all subfragments of the starting sample with respect to their nucleotide sequence.

The longer subfragments are subjected to another sample preparation cycle. This may again be an enzymic fragmentation or else a suitable amplification method or the previously described purely statistical POKS method and corresponding sample preparation.

If required, it is also possible to a use a plurality of enzyme POKS simultaneously in the sample preparation and in the subsequent cyclic array analysis. These subfragments may be assigned unambiguously by the enzymic POKS sequence at the start or the end of the probes and be monitored in parallel.

Due to predetermining the enzyme sequences, this variant of the DSBH method results in two possibilities for constructing the probes. Firstly, the complete sequence may be synthesized at the ends of the probes, and secondly it may be sufficient to synthesize only the part of the enzyme sequence downstream of the cleavage point. Table 7 illustrates the two possibilities via the example of a DNA sequence in which the sequence of the enzyme Alu I (AGCT) is present. The cleavage site of this enzyme is between the second and third nucleotide.

TABLE 7

5′ end NNNNNNNNNNNNN AG | CT NNNNNNNNNNNNNN 3′ end
3′ end NNNNNNNNNNNNN TC | CA NNNNNNNNNNNNNN 5′ end

After hydrolysis and denaturation during sample preparation, in this case four fragments are obtained. Two of them start, read in 5′-3′ direction, with the nucleotides CT and the other two ends with AG. In order to be able to identify the nucleotides following the enzyme sequence in both directions, the three above-described types of probe must then be synthesized on the array, see Table 8.

In the left half of Table 8, the complete enzyme sequence is used as POKS, and construction is carried out in complete analogy to the method using statistically chosen POKS. For constructing the probes listed in the right half, the enzyme sequence is split into two parts at its cleavage point. In order to be able to detect the fragments starting with the nucleotides CT in the above sequence example, probes having at the 3′ end the nucleotides GA are generated; in order to be able to determine the other two fragments, all probes of a predetermined length which carry at the 5′ end the nucleotides TC are generated. The hybridization behavior on the array must be identical for both types of probe. In the left case, the nucleotides TC act as a kind of linker.

For the in each case third type of probe, the sample must be prepared in a different way. The sequence to be investigated is either split statistically, for example with ultrasound, or cut using, for example, an enzyme whose sequence does not correspond to any of the enzyme sequences used for sample preparation.

TABLE 8

| | | | | | | | |
|---|---|---|---|---|---|---|---|
| N | A | N | 5′ end | N | C | N | 5′ end |
| N | G | N | N | T | N | | |
| N | C | N | N | N | N | | |
| N | T | N | N | N | N | | |
| N | N | N | N | N | N | | |
| N | N | N | N | N | N | | |
| N | N | A | N | N | A | | |
| N | N | G | N | N | G | | |
| N | N | C | N | N | C | | |
| N | N | T | N | N | T | | |
| N | N | N | N | N | N | | |
| A | N | N | N | N | N | | |
| G | N | N | N | N | N | | |
| C | N | N | A | N | N | | |
| T | N | N | 3′ end | G | N | N | 3′ end |

The individually detected fragments are assembled to a total sequence analogously to the described variant with statistically chosen POKS.

The essential advantage of generating the POKS in the sample preparation by cutting enzymes is a low requirement of sample material. The enzymic cleavage of the starting sequence produces only subfragments having the POKS sequence at the end. With a starting sequence of, for example, 3 000 bases and an average subfragment length of 60 bases, approx. 500 subfragments are produced. When splitting the same starting sequence into all possible subfragments of the freely selectable POKS (but with the same nucleotide sequence that the enzyme has), correspondingly 3 000−60+1=2 941 subfragments are produced, of which only 500 have the POKS sequence at the end. In comparison, the enzyme POKS thus require only 500/2 941=0.17, corresponding to 17% of the sample material.

The essential disadvantages of the enzymic POKS are the necessary development of suitable cutting enzymes, the low flexibility and the more complex sample preparation. The development of the appropriate enzymes, for example by means of protein design, is labor-intensive. The provision in the sample preparation increases the logistic complexity in the system. Moreover, a cyclic sample preparation with integrated length fractionation must be established. The latter is necessary in order to remove and further cut up the longer subfragments.

Both approaches (freely selectable and enzymic POKS) may also be combined. Thus it could be possible to provide statistically very successful POKS as enzymes in the sample preparation. If these enzyme POKS are used up, a correspondingly larger amount is amplified and the freely selectable POKS are used.

7.1.1 Freely Chosen POKS with all 3 Types of Probe

In this example, sequencing of a single-stranded start sequence of 3 060 nucleotides from the E. coli genome with the aid of various POKS of three nucleotides in length is simulated. The data generated during the simulation are ideal data which do not yet take into account possible errors such as, for example, a possible stop during synthesis or problems during the signal analysis.

The starting sequence can be assembled again in its entirety with the aid of the data generated by simulating the array construction, the hybridization and the signal analysis.

At the start of the process, the A-T, G-C content of the sequence is determined. Following this, the POKS with the highest probability, in this case GCG, is chosen as start POKS. This POKS is used to simulate the synthesis of the probes on the first array. For this purpose, all three types of probe having the POKS-complementary sequence at the positions in the probes, which are described in more detail above, are generated. In this example, the variable portion of the probes has a length of 5 nucleotides, and thus each type of probe requires positions, i.e. 3 072 in total. In order to utilize a possibly distinctly larger number of positions, it may be sensible to synthesize longer probes right at the start.

After the hybridization, in each case 82 positions whose probes have the POKS-complementary sequence at their ends and 81 positions whose probes have the POKS sequence in the center emit signals. Thus, on the next array a total of 980 (82×4+81×4+81×4) positions is required in order to be able to construct for each signal-emitting position four new positions with probes extended in each case by one base.

At this point, it is possible to process a plurality of iteration steps on one array at the same time, if the number of the positions present is sufficiently large. For this purpose, each relevant probe on the new array can be extended by two, three or more nucleotides. With an extension by two nucleotides, 16 new positions are then required per position, with an extension by three nucleotides correspondingly 64 positions are required, with 4 nucleotides 256 positions, etc. In the simulation in which the number of positions plays a minor part, a new array is generated for each iteration step.

In this case, the probe length of in total 5+3=8 nucleotides is already so specifically long that the number of the positions required does not increase in any of the subsequent iteration steps and settles down, after approximately 3 steps, to 340 positions per probe type, i.e. 1 020 positions in total.

In total, the probes are synthesized to a length of 25 nucleotides so that, after evaluating the last array, all 22-mers downstream and upstream of the first POKS, which are present in the starting sequence, are known. With the aid of the third type of probe, all possible connections between said part sequences are determined and these sequences can be arithmetically extended with the sequences of the first and second types of probe to in each case 47 nucleotides.

Thus, using the dynamic array construction has made it possible to determine all 22-mers downstream and upstream of the POKS without having to generate all 22-mers (4²²=1.759218604×10¹³).

In the next step, the now known assembled part sequences having the POKS in the center are searched for the POKS sequence to the right and to the left of said POKS. If the POKS sequence is found in a part sequence for a second time, the corresponding section is compared with all part sequences which have the POKS in the center. Since all sequences around the POKS are now known, there must be a sequence with which there is an overlap. After the first POKS, it is already possible to assemble the identified part sequences to longer sequences of up to 248 nucleotides in length. By analyzing the ends of said sequences, two new POKS (CTG, GAA) are determined, one for each end, which are then again used to construct arrays. As above, construction is started with a variable length of 5 nucleotides which is increased up to a length of 22 nucleotides. The number of positions required settles down, after a few cycles, to 312 per probe type so that a total of 936×2 positions per iteration step are required.

As before, the POKS sequences are searched in the detected sequences and these sequences are extended, where appropriate. After the first three POKS, it is possible to assemble sequence parts of up to a length of 456 nucleotides. In order to be able to identify and assemble the full-length sequence, four more POKS (GCC, CAG, TCA, ATC) are required, which are determined from the data analyzed thus far and another cycle. The number of positions required per iteration step in the last two cycles (array construction, hybridization, iterative extension of the probes up to 25 nucleotides) is at 200 to 370 positions per probe type. After the last cycle, the starting sequence can be completely assembled.

The array size and the number of POKS chosen after each step have not been optimized in this example. It is possible that a large number of POKS at the start of the method would reduce the number of positions/arrays required. Moreover, it seems sensible to process on each array a plurality of iteration steps at the same time in order to utilize the number of available positions. If, in this example, an array size of 400 000 positions is used as a starting point and the method is optimized, it is possible to construct on the first array probes with a variable part of 8 nucleotides, i.e. with a total length of 11 nucleotides. This, however, utilizes only half of the positions present, and this makes it seem sensible to choose two POKS at the start.

Even with a starting length of 11 nucleotides per probe, only approx. 85 positions per probe type emit signals so that on the next array a total of 1 020 positions must be constructed. Thus it is possible to process on this array 5 iteration steps, and for this 261 124 positions are required. Using two further arrays on which again in each case 1 024 probes per signal-emitting position of the preceding array can be constructed can extend the relevant probes to in each case 25 nucleotides. Thus, 4 arrays are required for the first POKS; in this case, the individual arrays are not yet used ideally.

In order to be able to investigate in the next steps two POKS at once, the number of iteration steps per array must be reduced to four so that for each POKS pair a total of four to five arrays is required, i.e. in total 16 to 19 arrays, inclusive of the arrays for the first POKS.

In examples with longer sequences it can be observed that the number of POKS required does not necessarily increase with the length of the sequence, but rather it is possible to assemble, for example, various sequences of 20 000 nucleotides in length having 9 to 11 POKS. The method thus becomes more and more economical for longer sequences.

8. Applications

The method of the invention makes possible the systematic sequence analysis of partially or completely unknown nucleic acids in a sample.

In one embodiment, the method is used to sequence genomes completely or partially. The parts may be generated by selecting and isolating individual chromosomes, by cloning genomic DNA (e.g. in Bacterial Artificial Chromosomes BAC or Yeast Artificial Chromosomes YAC) or by other methods.

In another embodiment, cDNA populations which may be prepared, for example, from a cloned library or directly from an isolated mRNA are completely or partially sequenced. The result then represents a transcriptome sequencing. This may be carried out, with simultaneous processing of different samples from different sources, for example cells in different states, such that in one variant only those sequences which are different and in another variant only those which are identical are pursued further.

In one embodiment it may be of interest that “polymorphisms”, for example single-nucleotide polymorphisms, are identified or used for selecting the POKS.

Furthermore it is possible to use the sequencing method of the invention for diagnostic purposes, for example for an individualized or multi-step diagnosis. The method is also suitable for developing an individualized, patient-dependent medication or for the patient-dependent development or/and modification of pharmaceutical substances. The methods may be used in connection with a network or/and a database for a decentralized analysis and identification close to the patient of disease states or pathogens and mutations thereof. Moreover, the method is suitable for molecular diagnostics and comparative genomics, for example for use in research, for elucidating the functionality of individual genes or genomes of organisms. Furthermore, the method may be used for mutation analysis, for example, inter alia, for investigating the influence of, for example, environmental factors, medicaments, radiation or/and venoms of organisms.

1. A method for sequencing nucleic acids, comprising the following steps:
(a) carrying out a first hybridization cycle, comprising
(i) providing a support having a surface which contains immobilized hybridization probes in a multiplicity of predetermined areas, said hybridization probes in individual areas having in each case a different base sequence of a predetermined length,
(ii) contacting a sample which contains nucleic acids to be sequenced with the support under conditions under which a hybridization between the nucleic acids to be sequenced and probes complementary thereto on the support can take place, and
(iii) identifying the predetermined areas on the support, in which a hybridization has taken place in step (ii),
(b) carrying out a subsequent hybridization cycle, comprising:
(i) providing a further support having a surface which contains immobilized hybridization probes in a multiplicity of predetermined areas, said hybridization probes in individual areas having in each case a different base sequence of a predetermined length, for said further support hybridization probes having a base sequence being selected for which in a preceding cycle a hybridization has been observed, and the selected hybridization probes being extended by at least one nucleotide compared with a preceding cycle,
(ii) repeating step (a) (i) using the further support, and
(iii) repeating step (a) (iii) using the further support, and
(c) carrying out, where appropriate, further subsequent hybridization cycles in each case with selection and extension and selection of the hybridization probes according to step (b) (i), until there is sufficient information about the nucleic acids to be sequenced.

2. The method as claimed in claim 1, characterized in that the nucleic acids to be sequenced are selected from the group consisting of double-stranded DNA, single-stranded DNA and RNA.

3. The method as claimed in either of claims 1 and 2, characterized in that the nucleic acids to be sequenced are fragmented prior to contacting the support.

4. The method as claimed in claim 3, characterized in that fragmentation and, where appropriate, subsequent fractionation by length generate nucleic acid fragments having a predetermined, for example essentially homogeneous, length distribution.

5. The method as claimed in either of claims 3 and 4, characterized in that the fragmentation is carried out sequence-unspecifically.

6. The method as claimed in either of claims 3 and 4, characterized in that the fragmentation is carried out sequence-specifically.

7. The method as claimed in any of the preceding claims, characterized in that the nucleic acids to be sequenced carry labeling groups, in particular optically detectable labeling groups such as fluorescent labels or metal particle labels.

8. The method as claimed in claim 7, characterized in that direct or indirect labels are used.

9. The method as claimed in any of the preceding claims, characterized in that in the first hybridization cycle probes having a length s are selected and all possible 4^{s} sequence variations are generated in the predetermined areas of the support.

10. The method as claimed in any of the preceding claims, characterized in that in the first hybridization cycle probes having a length s are selected such that, after contacting the sample, a hybridization with the nucleic acids to be sequenced takes place in not more than 25% of the predetermined areas.

11. The method as claimed in any of the preceding claims, characterized in that in the first hybridization cycle probes having a length s are selected such that they are related to the length m of the sequence to be determined in the following way:
m≦4^{s-1}+s−1

12. The method as claimed in any of the preceding claims, characterized in that in one or more hybridization cycles probes are used which, in addition to variable sections of the length n, have one or more, for at least part of the probes, fixed sections of the length p.

13. The method as claimed in claim 12, characterized in that in the first hybridization cycle the length n of the variable portion of the probe is selected such that all possible 4ⁿ sequence variations are generated in the predetermined areas of the support.

14. The method as claimed in either of claims 12 and 13, characterized in that the length p of the fixed section and the length n of the variable sections are selected such that they relate to the length m of the sequence to be determined in the following way:
m≦4ⁿ⁻¹ (4^{p}+p−1)

15. The method as claimed in any of claims 12 to 14, characterized in that the length of the fixed sections p is 2, 3 or 4 nucleotides.

16. The method as claimed in any of claims 12 to 15, characterized in that the probes used are selected from the group consisting of (1) probes having the fixed sections p on the 3′ end, (2) probes having the fixed sections p on the 5′ end and (3) probes having fixed sections p within the sequence.

17. The method as claimed in claim 16, characterized in that probes having fixed sections p within the sequence are used.

18. The method as claimed in either of claims 16 and 17, characterized in that the probes (1), (2) and (3) are employed together or/and successively on the same support or on different supports.

19. The method as claimed in any of claims 12 to 18, characterized in that the fixed sections p are determined at the start of the method or/and owing to the results of preceding hybridization cycles.

20. The method as claimed in any of claims 12 to 19, characterized in that the fixed sections are determined randomly, due to statistical considerations or/and due to biochemical considerations.

21. The method as claimed in any of claims 12 to 20, characterized in that the fixed sections are determined owing to the base sequence of enzyme or/and ribozyme recognition sequences, for example of nucleases.

22. The method as claimed in claim 21, characterized in that said enzymes are restriction endonucleases.

23. A support for sequencing nucleic acids, having a surface which contains immobilized hybridization probes in a multiplicity of predetermined areas, said hybridization probes in individual areas having in each case a different base sequence of a predetermined length, it being possible for said hybridization probes to have, in addition to variable sections of the length n, one or more, for at least part of the probes, fixed sections of the length p.

24. The support as claimed in claim 23, characterized in that it is a microfluidic support.

25. The use of the support as claimed in claim 23 or 24 in a method for sequencing nucleic acids.

26. The use of a method as claimed in any of claims 1 to 22 or of the support as claimed in claim 23 or 24 for sequencing genomes, chromosomes, plasmids, BACs or/and YACs.

27. The use of a method as claimed in any of claims 1 to 22, or of the support as claimed in claim 23 or 24 for transcriptome sequencing.

28. The use of a method as claimed in any of claims 1 to 22 or of the support as claimed in claim 23 or 24 for identifying polymorphisms.
